(19) United States
(12) Patent Application Publication
(10) Pub. No.: US 2003/0068357 A1
(43) Pub. Date: Apr. 10, 2003

(54) Food product for lowering cholesterol levels

(76) Inventors: Lisa A. Vala, Plymouth, MN (US); Daniel J. Lewandowski, Bloomington, MN (US); Ronald A. Heddleson, Plymouth, MN (US); Eric T. Gugger, Rogers, MN (US); Alicia A. Perdon, Maple Grove, MN (US)

Correspondence Address: GENERAL MILLS, INC., P.O. BOX 1113, MINNEAPOLIS, MN, 55440, US

(21) Appl. No.: 09/975,615

(22) Filed: Oct. 10, 2001

(51) Int. Cl. A61K 35/78; A61K 31/715; A61K 31/56; A61K 9/00

(52) U.S. Cl. 424/439; 424/750; 424/773; 424/738; 514/54; 514/170

(57) ABSTRACT

The present invention is a food product intended for consumers that is both nutritionally complete and well balanced and provides beneficial hypocholesterolemic activity. The food product includes a combination of soluble fiber component selected from the group consisting of psyllium, oat flour, oat bran, barley, beta-glucan, guar gum, beet pulp and pectin and a second beneficial component selected from the group consisting of beta sitosterol, stigmasterol, campesterol, ergosterol, beta sitostanol, campestanol, stigmastanol and fatty acid derivatives thereof and/or a combination of one or more of the above to generate the health benefit.

### CROSS REFERENCES TO RELATED APPLICATIONS

None.

### STATEMENT REGARDING FEDERALLY SPONSORED RESEARCH OR DEVELOPMENT

Not applicable.

### FIELD OF THE INVENTION

The present invention is related to a novel food composition that is used as a food product intended for consumers. The food product provides for beneficial hypocholesterolemic activity in a nutritionally balanced food that includes a combination of soluble fiber and a sterol or stanol or a fatty acid derivative thereof.

### BACKGROUND AND SUMMARY OF THE INVENTION

Today there are a number of pharmaceutical applications and dietary supplements relating to the treatment of high cholesterol levels. However, there appears to be a growing consensus as the population ages to avoid ingesting pill after pill or ingest some sort of dietary supplement to attain some perceived beneficial effect from such drug products or having to eat products aimed at consumers whose single intent is a health benefit, such products often ignore the taste or mouth feel or causes some undesirable results. Individuals may also wish to avoid certain risks and side effects associated with certain medications. As such, there remains a continuing interest in developing good tasting, well balanced, food products that provide both a beneficial health effect as well as a vehicle in which to deliver the benefit in a palatable and efficient manner to meet the changing needs of the population.

Cholesterol in humans comes from primarily two sources, the body's own production of cholesterol (endogenic) and dietary cholesterol. Typically, the average person consumes between 350-400 milligrams of cholesterol daily, while the recommended intake is around 300 milligrams. Increased dietary cholesterol consumption, especially in conjunction with a diet high in saturated fat intake, can result in elevated serum cholesterol. Elevated serum cholesterol is a well-established risk factor for heart disease and therefore there is a need to mitigate the undesired effects of cholesterol accumulation. High cholesterol levels are generally considered to be those total cholesterol levels at 200 milligrams and above or LDL cholesterol levels at 130 milligrams and above.

Lipoproteins contain specific proteins and varying amounts of cholesterol, triglycerides and phospholipids. There are three major classes of lipoproteins and they include very low density lipoproteins (“VLDL”), low density lipoproteins (“LDL”) and high density lipoproteins (“HDL”). The LDLs are believed to carry about 60-70% of the serum cholesterol present in an average adult. The HDLs carry around 20-30% of serum cholesterol with the VLDL having around 1-10% of the cholesterol in the serum. To calculate the level of non-HDL cholesterol present (find the level of LDL or VLDL levels), which indicates risk, the HDL is subtracted from the total cholesterol value. By lowering the total system LDL cholesterol level, it is believed that certain health risks, such as coronary disease and possibly some cancers, that are typically associated with high cholesterol levels, can be reduced.

Numerous studies relating to modifying the intestinal metabolism of lipids have been done to illustrate that such effects can reduce a high cholesterol level. This may be done by hampering the absorption of triglycerides, cholesterol or bile acids. It is believed that certain plant sterols and stanols lower serum cholesterol levels by reducing the absorption of dietary cholesterol and/or bile acids from the intestines.

Natural plant sterols are similar structurally to cholesterol except in the arrangement of the basic side chains. Absorption of plant sterols in the intestines is believed to be minimal at best and sterols/steroids are generally excreted in the bile and in the stool. Thus, the levels of plant sterols in the serum are relatively low since they are not absorbed by the body and are relatively quickly excreted. Where the amount of sterols is increased in an effort to obtain greater beneficial or health effects, the sterols still do not increase significantly in amount in the blood serum as the absorption capability, however limited it may be, is quickly exceeded.

The present invention is directed at avoiding the unpleasant task of having to take or ingest pharmaceutical or tolerate dietary supplements or the taste associated with some fortified or modified food products or cereal, by providing a balanced and complete set of macro and micronutrients in accordance with the U.S. recommended daily values (“USRDV” or “DV”). The food product of the present invention achieves a lowering of cholesterol levels through the unique combination of soluble dietary fiber and a sterol or stanol. In addition, the consumer receives a well balanced and nutrionally complete meal when ingesting the food product of the present invention.

The present invention is a well-balanced and nutritionally complete food product that is palatable and has a good mouth feel and texture so that consumers of the product are not limited solely to those having elevated cholesterol levels, i.e. those total cholesterol levels above 200 mg or those with LDL levels above 130 mg. Consumers with lower cholesterol levels, whether total cholesterol or LDL cholesterol levels, can maintain their average or “good range” cholesterol levels and it is believed, do not experience any adverse effect such as a further lowering of these levels.

Plant sterols, and its saturated derivative, stanols, are considered safe in that they are natural components of vegetable fats and oils. Likewise, soluble dietary fiber also is known to be a safe ingredient due to its long history in food supply. Individually, the two components have a certain health value or benefit, but heretofore it was unknown to include the two components into a single food product along with other nutritionally beneficial ingredients to achieve an improved high cholesterol lowering product that tastes good.

Soluble fiber typically remains undigested, except by colonic microflora present in the lower intestines. Soluble dietary fiber is believed to have a beneficial effect in the reduction of high serum cholesterol levels and reducing the risk associated with such elevated levels. In addition, soluble dietary fiber can have the additional beneficial effect of reduced constipation and improved regularity. However, too much fiber in the diet can create undesirable gastrointestinal side effects such as flatulence, diarrhea, and abdominal cramps, etc. leading consumers to stay away from food products that contain too much dietary fiber, regardless of any associated health benefits. While some consumers may not completely avoid such products, they also do not typically regularly use such products due to the problems enumerated above or alternatively, or in combination due to the unpleasant taste of such products. This illustrates some of the problems with prior solutions that were aimed at providing high fiber diets directed at lowering cholesterol levels, and highlights the need to create a more balanced solution that fits not only within more normal dietary patterns but also meets consumer demand for better tasting, healthy products.

There are a number of other products purporting to have cholesterol-lowering properties available in the market today. One such product offering or solution is described in U.S. Pat. No. 6,136,349 which relates to a food product, food additive or the like that may be fortified with a select group of minerals, such as calcium, magnesium or potassium which when combined with conventional sterols and/or stanols increases the effect of the sterols and/or stanols in lowering cholesterol levels than with just sterols an/or stanols alone. However, significantly increasing only certain nutrients and minerals while ignoring others can result in over consumption or under consumption of essential nutrients because some nutrients are present in very high concentrations while other nutrients are present in very low concentrations. This creates a nutritionally unbalanced situation causing the consumer to either procure the missing macro and/or micronutrients through other food sources or omit them from their diet altogether. In addition to not receiving the DV (Daily Value) of certain nutrients, this may force the consumer into an over compensation mode causing the consumer to ingest more food than is actually necessary thereby defeating the purpose of such cholesterol-lowering foods, and potentially create other problems such as weight gain.

Another possible solution is described in U.S. Pat. No. 6,174,560, which relates to a food composition for lowering low density cholesterol levels (LDL) and focuses on the use of at least one stanol fatty acid ester in combination with a nutritional substance. The applicants of U.S. Pat. No. 6,174,560 however indicates that increasing the amount of fiber to reduce serum cholesterol levels has been of a limited effect and citing that fiber that is delivered in therapeutically effective doses, such as with pharmaceutical applications, can cause extreme abdominal discomfort. This provides another singular example of a particular element or component being relied upon for a health effect but still ignoring the combined beneficial effects of the present invention as well as the ability to deliver the food product in an acceptable manner.

U.S. Pat. No. 5,244,887 describes the use of stanols as food additives to reduce cholesterol absorption. In the preparation of the additives, sitostanol is dissolved with an edible solubilizing agent such as triglyceride, an antioxidant such as tocopherol, and a dispersant such as lecithin, polysorbate 80, or sodium lauryl sulfate. However, no data is provided in the selection of the most effective components and their amounts or specific methods of preparation. Effectiveness in reducing cholesterol absorption was also not determined. The preferred embodiment consisted of 25% by weight stanols in vegetable oil, but the solubility of sterols in oil is only 2%.

Thus, there is a need to provide a nutritional food product that is balanced and complete as well as one that generates a health benefit, particularly one related to the reduction of harmful cholesterol levels in the blood, without the various side effects or challenges proposed in some of the past solutions.

A preferred embodiment of the present invention relates to a consumer food product having the beneficial effect of reducing high cholesterol levels while having little or no effect on cholesterol levels within acceptable ranges. The food product has at least one nutritionally beneficial soluble fiber component that is selected from the group consisting of psyllium, oat flour, oat bran, barley, beta-glucan, guar gum, beet pulp and pectin. At least a second nutritionally beneficial component selected from the group consisting of beta sitosterol, stigmasterol, campesterol, ergosterol, beta sitostanol, campestanol, stigmastanol and fatty acid derivatives thereof and/or a combination of one or more of the above is also provided. The combination of the two beneficial products, when ingested provides a serum cholesterol lowering effect.

A further embodiment of the present invention relates to a consumer food product for use in reducing LDL cholesterol levels in humans. The product includes at least a first soluble fiber component having a first beneficial health effect and at least a second sterol based component having a second beneficial health effect that is different from the first beneficial effect. The combination of the first and second components provided in a particular formula or ratio to create a product that delivers an enhanced health benefit of reducing LDL cholesterol levels when compared with either of the first and second health effects individually.

A still further embodiment of the present invention relates to a consumer food product for use in reducing total cholesterol levels. The food product includes at least a first soluble fiber component having a first beneficial health effect and at least a second sterol based component having a second beneficial health effect, which may be similar to but that is distinct in effect from the first beneficial health effect. The combination of the first and second components provided in a particular formula or ratio to create a product that delivers an enhanced health benefit of reducing total cholesterol levels when compared with either of the first and second health effects individually. The food product ratio ranges from roughly about equal parts of free sterols to dietary fiber to a formula or ratio of wherein approximately twice as much of one component is present when compared to the other component.

The present invention is intended to be used as a cereal, including ready to eat (RTE) cereals and cereal bars, dairy products such as yogurt, ice cream and other frozen novelties, bakery products such as dough or refrigerated dough, fruit snacks and other snack products. In addition, the present invention is contemplated for use in baking mixes, such as cake, brownie and other desert mixes, ready to eat meals, such as meals that contain pasta and the like.

### DETAILED DESCRIPTION OF PRESENTLY PREFERRED EXAMPLE ILLUSTRATIVE EMBODIMENTS

The soluble fiber component of the present invention is derived from a wide variety of grains and are composed of polysaccharides having a variety of structures. Fiber is generally resistant to human digestive enzymes, except for colonic microflora present in the lower intestines, and are known for their water and ion-binding capacity. The total dietary fiber content as used in the present invention is in the range of about 1 to 20%, preferably around 8 to 14% with a protein content of at least 8 to 9%.

Psyllium, as used in the present invention, is a known mucilaginous material derived from seeds from the plants of the Plantago genus, Plantago ovata, found in sub-tropical areas. The seeds are dark and shiny and have something of a concave shape to the exterior. Psyllium has been regularly used as a laxative to promote regular bowel function. Psyllium seed may be used in ground, dehusked or in whole form and represents a source of soluble dietary fiber. However, psyllium can have a coarse or rough texture making ingestion occasionally difficult, if the fiber component is not processed in a manner making it readily useable in a consumer food product..

Pectins are polymeric chains of partially methylated galacturonic acids that possess the ability to form a gel in water. Pectins as used in the present invention are traditionally extracted from apple and citrus fruits as well as beet pulp which may represent the preferred source.

Oat flour as used in the present invention is essentially heat-treated oat groats (hulled, crushed oats) or rolled oats that are ground on a hammer mill or other smooth rolls. There is no separation of the components during the processing of the flour.

Oat bran used in the present invention is produced by grinding clean oat groats or rolled oats and separating the resulting flour by suitable means, such as sieving, into fractions such that the oat bran fraction is not more then 50% of the original starting material. The separated fraction should have at least 5.5% of beta glucan (dry weight basis), and a total dietary fiber content of at least 16% (dry weight basis), so that at least one third of the total dietary fiber is soluble fiber.

Barley, as used in the present invention, is processed in a manner that resembles oats as set forth above, in that it consists of cleaning, hulling, sieving and then grinding. Waxy hulless barley has a higher dietary fiber content than most other sources of fiber and can range from 14 to 20% of the dry weight and have a beta glucan content of around 8 to 10%.

Guar gum is produced from the guar plant Cyanaposis tetragonolobus by milling the endosperm after removal of the hull and germ. Guar gum is a galactomannan with liner chains of D-mannopyranosyl units having side branching units of D-galactopyransose attached by (1→6) linkages. Guar gum has good water binding properties facilitating its use in food products.

Beta glucans, particularly grain beta glucans (oats and barley), are a known source of dietary fiber and have been included in food products that are used in weight control (beta glucans used as fat substitutes) and as cholesterol lowering additives. The beta glucans that are used in this invention can be naturally occurring or be chemically or enzymatically modified by altering the specific linkages. In addition physical modification of the beta glucans may be achieved by shearing. Beta glucans are obtained from milled cereal grains such as oats and barley (waxy, hulless barley being a particularly good source) in a manner discussed above and are then extracted from the milled grains into warm water and then the solids are removed from the solution.

In the present invention, the first component of this food product formula comes from the soluble fiber group identified above, including psyllium, oat flour, oat bran, barley, beta-glucan, guar gum, beet pulp and pectin. The soluble fiber portion of the present invention ranges from 1 to 25 percent by weight of the total suggested serving size of the product or roughly 55 grams of food product per serving. The preferred weight percentage is about 1 to 18 percent. The soluble fiber component in the examples set forth herein comprises about one to three grams per serving with about 1.4 to 1.6 grams per serving being used in the cereal formulas set forth below.

Sterols occur in natural fats and oils, particularly in vegetable oils. Unsaturated vegetable oils and non-animal fat oils, such as soybean oil, wheat germ oil, cottonseed oil, safflower oil, peanut oil, rice oil, canola oil and the like are well known sources of sitosterol, stigmasterol and campesterol as well as various other materials such as higher aliphatic alcohols. Tall oil is also a significant source of sitosterol and campesterol. The sterol of the present invention may be concentrated by any suitable method such as by (1) saponification, extraction and crystallization; (2) distillation or 3) high pressure propane extraction or obtained from a by-product stream such as from an oil deodorization process.

Stanols are the 5 alpha saturated derivatives of plant sterols and may be derived from similar sources and methods as described above and concentrated.

In the present invention, the second component of this food product formula comes from the sterol/stanol group identified above, including beta sitosterol, stigmasterol, campesterol, ergosterol, beta sitostanol, campestanol, stigmastanol and fatty acid derivatives thereof and/or a combination of one or more of the above. The sterol/stanol portion of the present invention ranges from 1 to 25 percent by weight of the total suggested serving size of the product or roughly 55 grams of food product per serving. The preferred weight percentage is about 1 to 18 percent. The sterol/stanol component in the examples set forth herein comprises about 0.4 to 3 grams per serving with about 0.6 to 1.6 grams per serving being used in the cereal formulas set forth below.

### EXAMPLE 1

In an exemplary embodiment of the present invention, the nutritionally complete food product, in this case a ready to eat (RTE) cereal, may include the following micro and macronutrients in connection with a low sterol based formula. In this example, about 1-1.5 gm of free sterols are used. The serving size is approximately 55 grams. The ratio of free sterols to dietary fiber ranges from about 1 to 1 to about 1 to 2, with other ratios or combinations possible, such as 1 to 1.5, 1.5 to 2, 2 to 1 and 2 to 1.5.

| | Recommended Daily Value (“DV”) |
|---|---|
| Total Fat | 5% |
| Saturated Fat | 3% |
| Cholesterol | 0% |
| Sodium | 10% |
| Potassium | 5% |
| Total Carbohydrate | 14% |
| Dietary Fiber | 17% |
| Protein | 10% |
| Vitamin A | 10% |
| Vitamin C | 70% |
| Calcium | 0% |
| Iron | 80% |
| Vitamin D | 10% |
| Vitamin E | 100% |
| Thiamin | 100% |
| Riboflavin | 80% |
| Niacin | 80% |
| Vitamin B6 | 80% |
| Folic Acid | 100% |
| Vitamin B12 | 100% |
| Phosphorus | 15% |
| Magnesium | 10% |
| Zinc | 80% |
| Copper | 4% |

This example is based on a 2,000 calorie diet and other food products such as cereal bars, fruit snacks, diary and bakery products, baking mixes and ready to eat meals may contain additional vitamins, nutrients and or minerals as well as potentially varying amounts of the macro and micro nutrients set forth in the instant example.

### EXAMPLE 2

In the second example the nutritionally complete food product, in this case a ready to eat (RTE) cereal, may include the following micro and macronutrients in connection with a high sterol based formula. In this example, about 1.5 -2 gm of free sterols are used. The serving size is approximately 55 grams. The ratio of free sterols to dietary fiber ranges from about 1 to 1 to about 1 to 2, with other ratios or combinations possible, such as 1 to 1.5, 1.5 to 2, 2 to 1 and 2 to 1.5.

| | Recommended Daily Value (“DV”) |
|---|---|
| Total Fat | 4% |
| Saturated Fat | 3% |
| Cholesterol | 0% |
| Sodium | 10% |
| Potassium | 5% |
| Total Carbohydrate | 14% |
| Dietary Fiber | 17% |
| Protein | 10% |
| Vitamin A | 10% |
| Vitamin C | 70% |
| Calcium | 0% |
| Iron | 80% |
| Vitamin D | 10% |
| Vitamin E | 100% |
| Thiamin | 100% |
| Riboflavin | 80% |
| Niacin | 80% |
| Vitamin B6 | 80% |
| Folic Acid | 100% |
| Vitamin B12 | 100% |
| Phosphorus | 15% |
| Magnesium | 10% |
| Zinc | 80% |
| Copper | 4% |

This example is based on a 2,000 calorie diet and other food products such as cereal bars, fruit snacks, diary and bakery products baking mixes and ready to eat meals may contain additional vitamins, nutrients and or minerals as well as potentially varying amounts of the macro and micro nutrients set forth in the instant example.

The RTE cereals of the preceding examples are prepared in a conventional manner. This exemplary RTE cereal is in the form of flakes that are created by preparing a cooked cereal dough through known methods and then forming the cooked cereal dough into pellets that have a desired moisture content. The pellets are then formed into wet flakes by passing the pellets through chilled roller and then subsequently toasting or heating the wet cereal flakes. The toasting causes a final drying of the wet flakes, resulting in slightly expanded and crisp RTE cereal flakes. The flakes are then screened for size uniformity. The final flake cereal attributes of appearance, flavor, texture, inter alia, are all affected by the selection and practice of the steps employed in their methods of preparation. For example, to provide flake cereals having a desired appearance feature of grain bits appearing on the flakes, one approach is to topically apply the grain bits onto the surface of the flake as part of a coating that is applied after toasting.

It will thus be seen according to the present invention that a highly advantageous food product foe lowering high cholesterol levels has been provided. While the invention has been described in connection with what is presently considered to be the most practical and preferred embodiment, it will be apparent to those of ordinary skill in the art that the invention is not to be limited solely to the disclosed embodiment, that many modifications and equivalent arrangements may be made thereof within the scope of the invention, which scope is to be accorded the broadest interpretation of the appended claims so as to encompass all equivalent structures and products.

1. A consumer food product having the beneficial effect of reducing high cholesterol level ranges comprising;
at least a first beneficial soluble fiber component selected from the group consisting of psyllium, oat flour, oat bran, barley, beta-glucan, guar gum, beet pulp and pectin;
at least a second beneficial component selected from the group consisting of beta sitosterol, stigmasterol, campesterol, ergosterol, beta sitostanol, campestanol, stigmastanol and fatty acid derivatives thereof; and
wherein ingestion of the consumer food product containing a combination of said first and second beneficial components provides a serum cholesterol lowering effect.

2. A consumer food product as recited in claim 1 wherein said first beneficial component comprises between 1 to 25 percent by weight.

3. A consumer food product as recited in claim 1 wherein said second beneficial component comprises between 1 to 25 percent by weight.

4. A consumer food product as recited in claim 1 wherein said consumer food product is a baking mix.

5. A consumer food product as recited in claim 1 wherein said consumer food product is a ready to eat meal.

6. A consumer food product as recited in claim 1 wherein said consumer food product is a cereal.

7. A consumer food product as recited in claim 1 wherein said consumer food product is a ready to eat cereal.

8. A consumer food product as recited in claim 1 wherein said consumer food product is a cereal bar.

9. A consumer food product as recited in claim 1 wherein said consumer food product is a refrigerated dough.

10. A consumer food product as recited in claim 1 wherein said consumer food product is a fruit snack.

11. A consumer food product as recited in claim 1 wherein said consumer food product is a dairy based product.

12. A consumer food product as recited in claim 11 wherein said dairy based product is a yogurt.

13. A consumer food product as recited in claim 11 wherein said dairy based product is an ice cream.

14. A consumer food product as recited in claim 11 wherein said dairy based product is a drinkable product.

15. A consumer food product as recited in claim 1 wherein said consumer food product is a bakery product.

16. A consumer food product as recited in claim 1 wherein said consumer food product is a snack product.

17. A consumer food product for use in reducing LDL cholesterol levels, comprising;
at least a first soluble fiber component having a first beneficial health effect selected from the group consisting of psyllium, oat flour, oat bran, barley, beta-glucan, guar gum, beet pulp and pectin;
at least a second sterol based component having a second beneficial health effect, which may be similar to said first beneficial health effect;
said at least first and second components provided in a particular ratio to create a food product that delivers a health benefit of reducing LDL cholesterol levels.

18. A consumer food product as provided in claim 17 wherein said first soluble fiber component is provided in at least an equal amount to said second sterol component.

19. A consumer food product as provided in claim 17 wherein said first soluble fiber component is provided in an amount of approximately 1 to 3 grams per serving size.

20. A consumer food product for use in reducing total cholesterol levels, comprising;
at least a first soluble fiber component having a first beneficial health effect selected from the group consisting of psyllium, oat flour, oat bran, barley, beta-glucan, guar gum, beet pulp and pectin;
at least a second sterol based component having a second beneficial health effect, which may be similar to but distinct in effect from said first beneficial health effect;
said at least first and second components provided in a particular ratio of one to another to create a food product that delivers a health benefit of reducing total cholesterol levels.
